(19) United States
(12) Patent Application Publication
(10) Pub. No.: US 2026/0083788 A1
(43) Pub. Date: Mar. 26, 2026

(54) ANTIVIRAL FUNGAL EXTRACTS

(71) Applicant: Mycotech Pharma AS, Oslo (NO)

(72) Inventors: Ralf Schmidt, Nesbru (NO); Bernd Fiebich, Freiburg (DE)

(73) Assignee: Mycotech Pharma AS, Oslo (NO)

(21) Appl. No.: 19/106,961

(22) PCT Filed: Jul. 21, 2023
(86) PCT No.: PCT/EP2023/070290

§ 371 Date: Feb. 26, 2025

(30) Foreign Application Priority Data
Aug. 29, 2022 (EP) 22192650.4
Jun. 12, 2023 (NO) 20230677

(51) Int. Cl. A61K 36/07; A61K 9/00; A61K 36/18; A61P 31/14

(52) U.S. Cl. CPC A61K 36/071; A61K 9/006; A61K 9/0073; A61K 36/07; A61K 36/18; A61P 31/14

(57) ABSTRACT

The present disclosure provides compositions able to prevent coronavirus infections. As demonstrated by the examples herein, compositions comprising an extract from Agaricus blazei, Grifola frondosa and Hericium erinaceus may interfere with the interaction between the Spike S1 protein and ACE2. Accordingly, such compositions may provide therapeutic or prophylactic alternatives to the known treatments.

### FIELD

The present disclosure concerns mushroom extracts and their use in antiviral drugs or food supplements.

### BACKGROUND

Coronavirus disease 2019 (COVID-19) increases the risk of developing Acute Respiratory Distress Syndrome (ARDS), which is often fatal at the late stages of the infection when SARS-CoV-2 causes significant damage to the lungs. Several COVID-19 Vaccines have been approved or Authorized for Emergency Use by FDA. They include:
Comirnaty and Pfizer-BioNTech COVID-19 Vaccine
Spikevax and Moderna COVID-19 Vaccine
Janssen COVID-19 Vaccine
Novavax COVID-19 Vaccine, Adjuvanted

The FDA has approved the antiviral drug Veklury (remdesivir) for adults and certain pediatric patients with COVID-19, but this is an intravenous therapy. The FDA has also approved the immune modulator Olumiant (baricitinib) for certain hospitalized adults with COVID-19.

However, other alternatives would be useful, in particular drugs for oral administration or even non-drug alternatives such as food supplements.

### SUMMARY

The present disclosure provides compositions able to treat, prevent or ameliorate coronavirus infections. As demonstrated by the examples herein, compositions comprising an extract from Agaricus blazei, Grifola frondosa and Hericium erinaceus may interfere with the interaction between the SARS-CoV-2 Spike S1 protein and membrane bound Angiotensin Converting Enzyme 2 (ACE2). When combined with dissolved powder from Cistus creticus or an aqueous extract from Cistus creticus, a synergistic effect is seen. Accordingly, such compositions may provide therapeutic or prophylactic alternatives to the known treatments. Furthermore, such compositions are also demonstrating inhibiting effects on neuroaminidase. This enzyme is an attractive target for antiviral strategies because of its essential role in the pathogenicity of many respiratory viruses. These discovered mechanisms open up oral, intranasal and/or pulmonary administration as attractive implementation. Accordingly, devices comprising the compositions disclosed herein may provide sprays reaching epithelial cells where corona-viruses may be prevented from entering the cells and other respiratory viruses (e.g. influenza-virus or rhino-viruses) may be prevented from being released from infected cells.

In a first embodiment, we provide a pharmaceutically acceptable composition comprising
a. an extract from Agaricus blazei,
b. an extract from Grifola frondosa, and/or
c. an extract from Hericium erinaceus
for use in treatment of coronavirus infections.

In a first aspect of the first embodiment, the composition comprises
an extract from Agaricus blazei,
an extract from Grifola frondosa, and
an extract from Hericium erinaceus.

In a second aspect of the first embodiment, the treatment is therapeutic or prophylactic.

In a third aspect of the first embodiment, the coronavirus is SARS-CoV-2.

In a fourth aspect of the first embodiment, the composition is an aqueous solution.

In a fifth aspect of the first embodiment, the composition is administered orally, intranasally or pulmonary.

In a sixth aspect of the first embodiment, the composition comprises an extract obtained by aqueous extraction of a mixture comprising 50 to 90 wt % mycelium from Agaricus blazei.

In a seventh aspect of the first embodiment, the composition comprises an extract obtained by aqueous extraction of a mixture comprising 10 to 50 wt % mycelium from Grifola frondosa.

In an eighth aspect of the first embodiment, the composition comprises an extract obtained by aqueous extraction from Hericium erinaceus stalks.

In a ninth aspect of the first embodiment, the extracts are obtained by aqueous extraction of mycelium from Agaricus blazei, mycelium from Grifola frondosa and Hericium erinaceus stalks.

In a tenth aspect of the first embodiment, the extract from Agaricus blazei is an aqueous solution comprising 2 g to 3.6 g dry matter material per liter.

In an eleventh aspect of the first embodiment, the extract from Grifola frondosa is an aqueous solution comprising 0.04 g to 0.8 g dry matter material per liter.

In a twelfth aspect of the first embodiment, the extract from Hericium erinaceus is an aqueous solution comprising 0.4 g to 1.2 g dry matter material per liter.

In a thirteenth aspect of the first embodiment, the pH is the range of 5 to 8.

In a fourteenth aspect of the first embodiment, the composition comprises an extract from Cistus creticus.

Any one of the aspects of the first embodiment, can be implemented in combination. For example, the first and second aspect may be implemented in combination, the first and third aspect may be implemented in combination, and of course, the first, second and third aspect may be implemented in combination.

In a second embodiment, we provide a method of treatment of a coronavirus infection, comprising the step of administering a pharmaceutically acceptable composition to a subject in need thereof, wherein the pharmaceutically acceptable composition comprises
a. an extract from Agaricus blazei,
b. an extract from Grifola frondosa, and/or
c. an extract from Hericium erinaceus.

In a first aspect of the second embodiment, the composition comprises
an extract from Agaricus blazei,
an extract from Grifola frondosa, and
an extract from Hericium erinaceus.

In a second aspect of the second embodiment, the treatment is therapeutic or prophylactic.

In a third aspect of the second embodiment, the coronavirus is SARS-CoV-2.

In a fourth aspect of the second embodiment, the composition is an aqueous solution.

In a fifth aspect of the second embodiment, the composition is administered orally, intranasally or pulmonary.

In a sixth aspect of the second embodiment, the composition comprises an extract obtained by aqueous extraction of a mixture comprising 50 to 90 wt % mycelium from Agaricus blazei.

In a seventh aspect of the second embodiment, the composition comprises an extract obtained by aqueous extraction of a mixture comprising 10 to 50 wt % mycelium from Grifola frondosa.

In an eighth aspect of the second embodiment, the composition comprises an extract obtained by aqueous extraction from Hericium erinaceus stalks.

In a ninth aspect of the second embodiment, the extracts are obtained by aqueous extraction of mycelium from Agaricus blazei, mycelium from Grifola frondosa and Hericium erinaceus stalks.

In a tenth aspect of the second embodiment, the extract from Agaricus blazei is an aqueous solution comprising 2 g to 3.6 g dry matter material per liter.

In an eleventh aspect of the second embodiment, the extract from Grifola frondosa is an aqueous solution comprising 0.04 g to 0.8 g dry matter material per liter.

In a twelfth aspect of the second embodiment, the extract from Hericium erinaceus is an aqueous solution comprising 0.4 g to 1.2 g dry matter material per liter.

In a thirteenth aspect of the second embodiment, the pH is the range of 5 to 8.

In a fourteenth aspect of the second embodiment, the composition comprises an extract from Cistus creticus.

Any one of the aspects of the second embodiment, can be implemented in combination. For example, the first and second aspect may be implemented in combination, the first and third aspect may be implemented in combination, and of course, the first, second and third aspect may be implemented in combination.

In a third embodiment, we provide a method for improving the immune system readiness in a subject, comprising the step of consuming a composition comprising
a. an extract from Agaricus blazei
b. an extract from Grifola frondosa, and/or
c. an extract from Hericium erinaceus.

In a first aspect of the third embodiment, the composition comprises
an extract from Agaricus blazei,
an extract from Grifola frondosa, and
an extract from Hericium erinaceus.

In a second aspect of the third embodiment, the composition comprises an extract obtained by aqueous extraction of a mixture comprising 50 to 90 wt % mycelium from Agaricus blazei.

In a third aspect of the third embodiment, the composition comprises an extract obtained by aqueous extraction of a mixture comprising 10 to 50 wt % mycelium from Grifola frondosa.

In a fourth aspect of the third embodiment, the composition comprises an extract obtained by aqueous extraction from Hericium erinaceus stalks.

In a fifth aspect of the third embodiment, the immune system readiness is improved with respect to coronavirus challenges.

In a sixth aspect of the third embodiment, the composition is an aqueous solution.

In a seventh aspect of the third embodiment, the subject is a healthy human.

In an eighth aspect of the third embodiment, the composition is consumed daily.

In a nineth aspect of the third embodiment, the composition is in the form of a food supplement, nutraceutical or functional food.

In a nineth aspect of the third embodiment, the composition comprises an extract from Cistus creticus.

Any one of the aspects of the third embodiment, can be implemented in combination. For example, the first and second aspect may be implemented in combination, the first and third aspect may be implemented in combination, and of course, the first, second and third aspect may be implemented in combination.

In a fourth embodiment, we provide compositions comprising
a. an extract from Agaricus blazei,
b. an extract from Grifola frondosa,
c. an extract from Hericium erinaceus, and
d. an extract from Cistus creticus.

In a first aspect of the fourth embodiment, the composition comprises an extract obtained by aqueous extraction of a mixture comprising 50 to 90 wt % mycelium from Agaricus blazei.

In a second aspect of the fourth embodiment, the composition comprises an extract obtained by aqueous extraction of a mixture comprising 10 to 50 wt % mycelium from Grifola frondosa.

In a third aspect of the fourth embodiment, the composition comprises an extract obtained by aqueous extraction from Hericium erinaceus stalks.

In a fourth aspect of the fourth embodiment, the extracts are obtained by aqueous extraction of mycelium from Agaricus blazei, mycelium from Grifola frondosa and stalks from Hericium erinaceus.

In a fifth aspect of the fourth embodiment, the pH is the range of 5 to 8.

In a sixth aspect of the fourth embodiment, the composition is aqueous and/or the extracts are aqueous.

In a seventh aspect of the fourth embodiment, the composition is sterile.

In a eighth aspect of the fourth embodiment, the composition is suitable for intranasal administration, pulmonary administration or for administration to the oral cavity.

In a ninth aspect of the fourth embodiment, the composition is classified as novel food, food supplement, nutraceutical or functional food.

Any one of the aspects of the fourth embodiment, can be implemented in combination. For example, the first and second aspect may be implemented in combination, the first and third aspect may be implemented in combination, and of course, the first, second and third aspect may be implemented in combination.

In a fifth embodiment, we provide devices for oral, intranasal or pulmonary administration comprising any of the compositions or aspects of the fourth embodiment.

In a first aspect of the fifth embodiment, the device comprises a nozzle configured to provide a spray of liquid particles able to reach pulmonary surfaces.

In a second aspect of the fifth embodiment, the device comprises a nozzle configured to provide a spray of liquid particles able to reach intranasal surfaces.

In a third aspect of the fifth embodiment, the device comprises a nozzle configured to provide a spray of liquid particles able to reach surfaces in the oral cavity.

Any one of the aspects of the fifth embodiment, can be implemented in combination. For example, the first and second aspect may be implemented in combination, the first and third aspect may be implemented in combination, and of course, the first, second and third aspect may be implemented in combination.

In a sixth embodiment, we provide the compositions of the fourth embodiment for use in treatment or prevention of coronavirus infections, rhinovirus infections or influenza infections by oral, intranasal or pulmonary administration.

In a first aspect of the sixth embodiment, the administration is in the form a suitable spray.

### BRIEF DESCRIPTION OF THE FIGURES

FIG. 1 visualizes the effect of Composition A on HEK-Blue-ACE2 cells challenged by a SARS-CoV-2 pseudotyped virus.
=p 0.05 vs control untreated cells, **=p 0.01 vs control untreated cells.
The infection level in the control sample, i.e. without Composition A, was set to 100%. Four samples with increasing amount of Composition A demonstrate decreased infection efficacy. The samples 1, 2, 3 and 4 contained 0.1 vol %, 1 vol %, 5 vol % and 10 vol % of Composition A, respectively.

FIG. 2 visualizes the effect of Composition A on the binding of SARS-CoV-2 Spike S1 protein to ACE2 ex vitro. The control sample did not contain Composition A. Sample 1, 2, 3, 4 and 5 contained 0.01 vol %, 0.1 vol %, 1 vol %, 5 vol % and 10 vol % of Composition A, respectively. Positive controls contained 10 nM or 100 nM of an antibody known to prevent the binding of Spike S1 protein to ACE2.

FIG. 3 visualizes the effects of Andosan and Cistus creticus extract separately or in combination on viral recombinant activity. These extracts inhibited neuraminidase (NA) activity in a concentration dependent manner. The results are expressed as percent of inhibition compared to NA alone, * * * p<0.001, ** p<0.01. The samples contained 4.16×10⁸ Units Neuraminidase from Influenza A Virus. Accordingly, in FIG. 3, 4, 16×10-8U means 4.16×10⁸ Units Neuraminidase from Influenza A Virus.

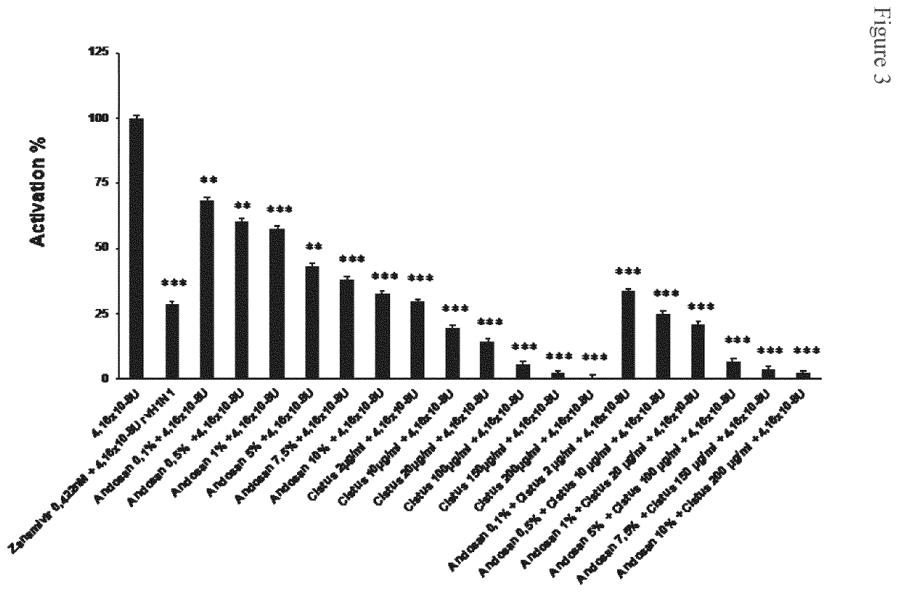

FIG. 4 visualizes the effects of Andosan and Cistus creticus extract separately or in combination on pseudotyped SARS-Cov2 infection. These extracts inhibited SARS-CoV-2 infection. The results are expressed as percent of inhibition compared to infection alone, * * * p<0.001, ** p<0.01, *p<0.05

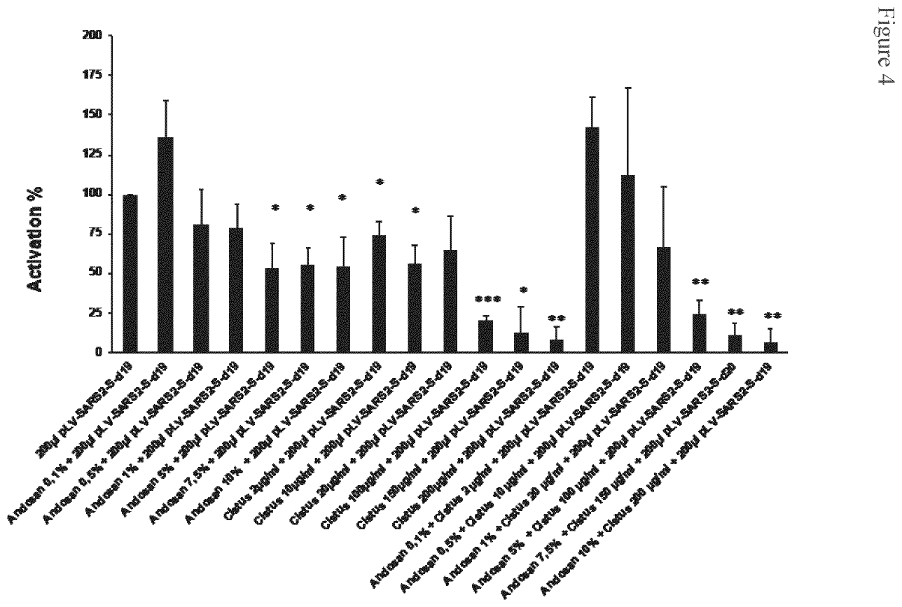

FIG. 5 visualizes the effects of Andosan and Cistus creticus extract separately or in combination on cell viability.

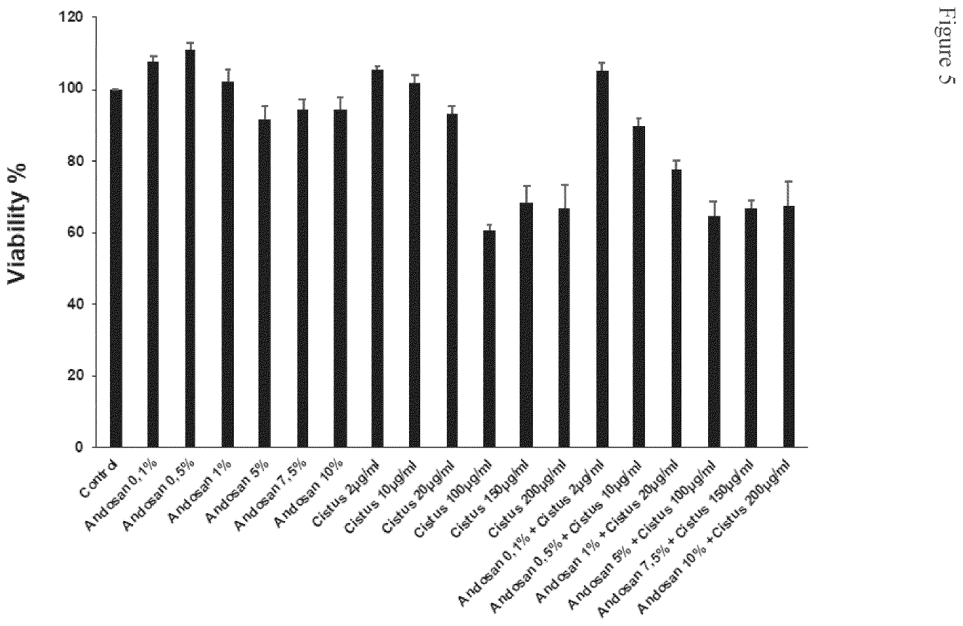

FIG. 6 visualizes the effect of Andosan on binding of SARS-CoV-2 Spike S1 protein to ACE2 ex vitro. The binding in the control sample was set to 100%. Samples with more than 5% Andosan demonstrate decreased binding of SARS-CoV-2 Spike S1 protein to ACE2.

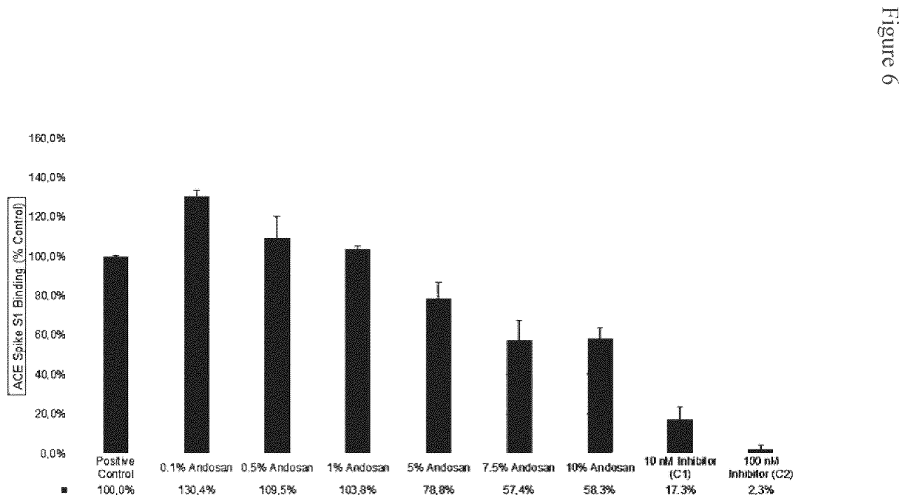

FIG. 7 visualizes the effect of an extract from Cistus creticus on binding of SARS-CoV-2 Spike S1 protein to ACE2 ex vitro. The binding in the control sample was set to 100%. Samples with more than 100 μg/ml dissolved Cistus creticus powder demonstrate decreased binding of SARS-CoV-2 Spike S1 protein to ACE2.

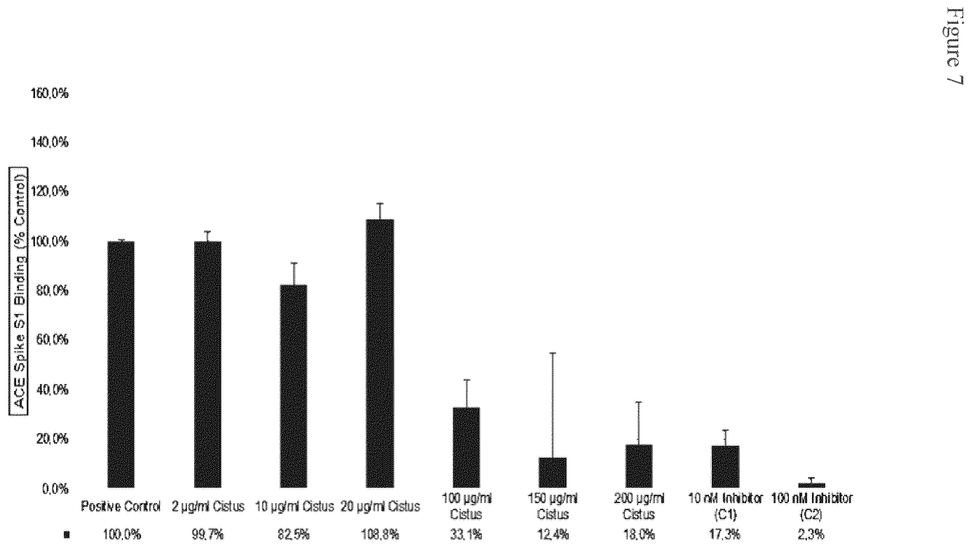

FIG. 8 visualizes the effect of Andosan comprising an extract from Cistus creticus on binding of SARS-CoV-2 Spike S1 protein to ACE2 ex vitro. The binding in the control sample was set to 100%. The combination of Andosan/Cistus shows synergistic effects (additive in the 100 μg/ml dose of Cistus). The combination of Andosan (1, 5, 10 vol %) with dissolved Cistus creticus powder (100 μg/ml) was more potent as Cistus or Andosan (1, 5, 10 vol %) alone. The combination of 10 vol % Andosan and 100 μg/ml Cistus was as potent as the positive control (10 nM/Spike S1 Neutralizing antibody)

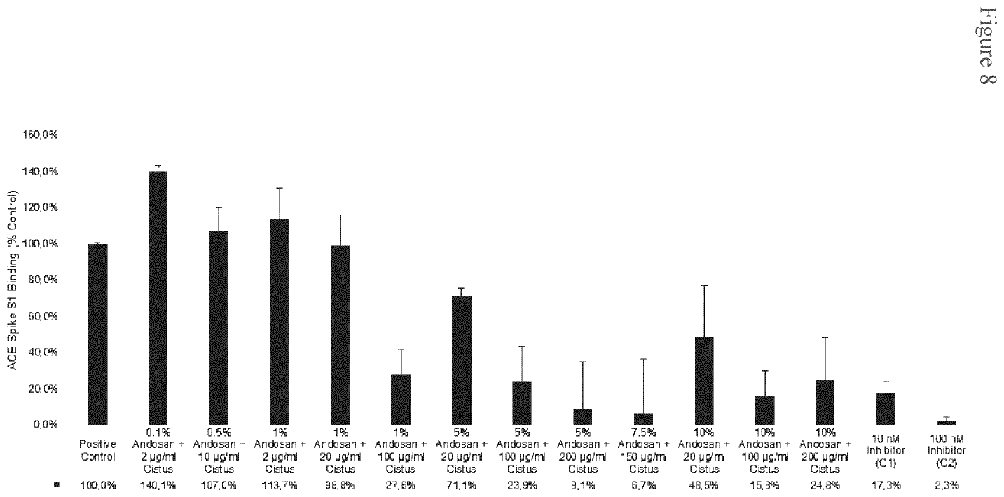

FIG. 9 visualizes a synergistic effect of Andosan comprising an extract from Cistus creticus on binding of SARS-CoV-2 Spike S1 protein to ACE2 ex vitro. The binding in the control sample was set to 100%. The combination of Andosan with Cistus shows synergistic effects (20 and 100 μg/ml dissolved Cistus creticus powder), even if the samples contain only 1 vol % Andosan.

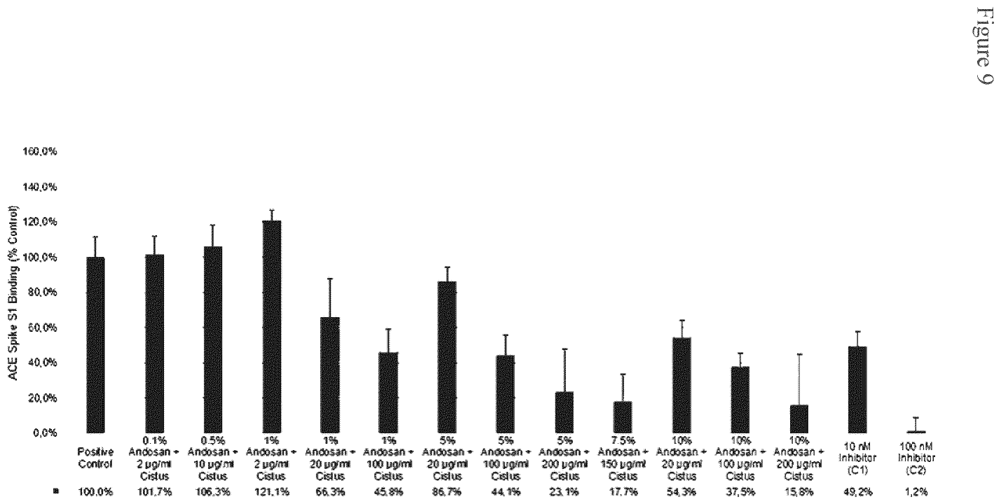

FIG. 10 visualizes a synergistic effect of Andosan comprising an extract from Cistus creticus on binding of SARS-CoV-2 Spike S1 protein to ACE2 ex vitro. The binding in the control sample was set to 100%. The combination of 10 vol % Andosan with Cistus (20 and 100 μg/ml dissolved Cistus creticus powder) reveals synergistic effects.

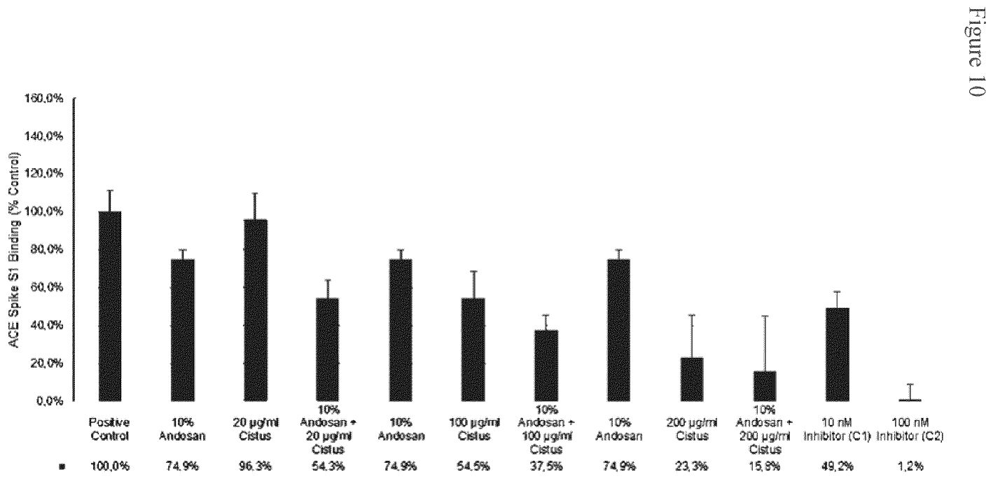

### DETAILED DESCRIPTION

Coronavirus (CoV) is a family of enveloped single-stranded RNA viruses, which are infectious to animals and people. The viruses are able to cause respiratory, hepatic, enteric, and neurological diseases of various severity. At least six species of coronaviruses are known to infect humans. A group of four such coronaviruses produce symptoms that are generally mild like common cold, e.g.
Human coronavirus OC43
Human coronavirus HKU1
Human coronavirus 229E
Human coronavirus NL63

However, three human coronaviruses are known to produce potentially severe symptoms:
Severe acute respiratory syndrome coronavirus (SARS-CoV),
Middle East respiratory syndrome-related coronavirus (MERS-CoV)
Severe acute respiratory syndrome coronavirus 2 (SARS-CoV-2)

The latter three may cause the diseases commonly called SARS, MERS, and COVID-19 respectively.

It is currently believed that, as a first step of the viral replication strategy, coronaviruses attach to the host cell surface before entering the cell. The interaction between coronaviruses and host cells is believed to involve the viral Spike S1 protein and the ACE2 in the host. ACE2 is found on the surface of type I and II pneumocytes, endothelial cells, and ciliated bronchial epithelial cells. Drugs preventing the interaction between the Spike S1 protein of SARS-CoV-2 and ACE2 may thus offer some protection against the viral infection.

It is found that compositions comprising an extract from Agaricus blazei, Grifola frondosa and Hericium erinaceus inhibited SARS-CoV-2 infections of HEK-cells expressing ACE2. Accordingly, it is believed that such extracts prevent the interaction between the Spike S1 protein and ACE2.

One such composition is AndoSan™ which is an aqueous solution comprising a heat-sterilized extract of the of Agaricus blazei Murill, Hericium erinaceus and Grifola frondosa. According to Hetland et al 2016 PLoS One; 11(12): e0167754, Andosan™ is a mixed Basidiomycetes mushroom water extract of the mycelium of (82.4%), Agaricus blazei Murill, Hericium erinaceus (14.7%) and Grifola frondosa (2.9%). It has a dry matter content of ca 4 g per 1 and it is marketed by ImmunoPharma AS in Norway.

Several methods for culturing of Agaricus blazei are known. One example is disclosed in U.S. Pat. No. 5,048,227 (OKUBO JUNYA), and its disclosure is incorporated herein.

Methods for aqueous extraction from Agaricus blazei Murill are disclosed in US20020119164 (UCHIYAMA SHOJI), and its disclosure is incorporated herein. The extracts in the present disclosure are solutions obtainable by subjecting fruitbody-material and/or mycelium to water, an aqueous solution, a polar non-toxic solvent and/or mixtures of these.

The correct botanical name for “Agaricus blazei” and “Agaricus blazei Murill” may be “Agaricus subrufescens”. Accordingly, when used in the present disclosure, they all mean the same.

The extraction can be done by immersing mushrooms or parts thereof (e.g. mycelium, spores, cap, stipe, annulus, gill/lamellae, basidia, filaments), in any or all stages of the life cycle, whether whole or particulate (e.g. chopped or ground to powder), in water, an aqueous solution, a polar non-toxic organic solvent and/or mixtures of these. The so obtained extract is an aqueous solution if water or an aqueous solution is used for extraction. The extraction process may be performed over hours, days, weeks or months. Continuous stirring with aeration may be beneficial for the activity of the filtered composition.

Suitable temperature of the extraction-solution may be between about 25° C. and 100° C.

Accordingly, suitable extracts may be obtained from the respective fruitbodies, the respective mycelium or from mixtures comprising both fruitbody-material and mycelium. Fruitbody-material may include, but is not limited to, cap-material, material from a spore-forming parts and/or stalk-material. Such extracts are preferably filtered to remove solid material.

The compositions herein may comprise extracts from Cistus creticus. “Cistus creticus”, as used herein, has its conventional meaning, and accordingly, it covers the subspecies eriocephalus (viv.) and it is a synonym for Cistus incanus subspecies tauricus. Aqueous extracts from Cistus creticus are commercially available from Finzelberg GmbH. Such extracts may be dried to form powders, and the powders may be dissolved in suitable solvents. When such powders are dissolved, an extract from Cistus creticus is (re)formed. When such powders are dissolved in water, an aqueous extract from Cistus creticus is (re)formed. All the compositions disclosed herein may comprise an aqueous extract from Cistus creticus. In particular, they may comprise 0.5 to 10 wt % dissolved powder from Cistus creticus. For example, a composition based on Andosan can comprise 0.5 to 10 wt % dissolved powder from Cistus creticus and have a viscosity suitable for conventional spray-pump devices for spraying onto the surfaces of the oral cavity.

Cistus extracts and nasal sprays are known, and a suitable production process is described in US2010062068 (KREWEL MEUSELBACH GMBH). The extract may be obtained from the aerial parts of the Cistus creticus. The plant parts may be subjected to extraction immediately after harvest, i.e., in a raw state. Alternatively, the plant parts are dried before the extraction. Subsequently, the leaves of the plant are suitably comminuted, for example, by attrition or cutting.

The extraction may be performed with a suitable solvent. Suitable solvents include aqueous or organic solvents, especially water, alcohols, such as methanol, ethanol or isopropanol, or chlorinated solvents, such as dichloromethane, and acetone, acetylacetone, ammonia, carbon dioxide or glacial acetic acid. Mixtures of the mentioned solvents may also be employed. Preferably, a mixture of water with methanol or ethanol is employed.

The extraction is usually performed at room temperature. However, it is also possible to perform the extraction at elevated temperatures of from 25° C. to the boiling point of the solvent employed. Extraction at room temperature is preferred.

In order to achieve as high a yield as possible, the plant material can be extracted several times. Also, different solvents may be employed in the different extraction steps.

Aqueous extracts from Cistus creticus and their production are also disclosed in US2011059190 (FINZELBERG GMBH).

When administration to human patients is intended, sterilization is likely needed, and it can be achieved by heat-treatment or other known sterilization methods.

The compositions in the present disclosure may thus be obtained by aqueous extraction from mixtures comprising mycelium from Agaricus blazei Murill in the range of 40 to 99 wt %, 50 to 95 wt %, 55 to 90 wt %, 60 to 85 wt % or 75 to 85 wt %. Said mixtures may also comprise mycelium from Grifola frondosa in the range of 1 to 30 wt %, 2 to 25 wt %, 5 to 20 wt % or 10 to 18 wt %. Preferably, such compositions may be mixed with aqueous extracts from Hericium erinaceus stalks.

It is of course possible to make the suitable compositions by mixing aqueous extracts from Agaricus blazei Murill, Grifola frondosa and Hericium erinaceus in any order.

Accordingly, such suitable compositions may for example contain a major fraction of an aqueous extract from Agaricus blazei Murill, and a minor fraction of aqueous extract from Grifola frondosa, and a minor fraction of an aqueous extracts from Hericium erinaceus.

Accordingly, such suitable compositions may for example contain a major fraction of an aqueous extract from Agaricus blazei Murill mycelium, and a minor fraction of aqueous extract from Grifola frondosa mycelium, and a minor fraction of an aqueous extract from Hericium erinaceus stalks.

Accordingly, such suitable compositions may for example contain a major fraction of an aqueous extract from Agaricus blazei Murill, and a minor fraction of an aqueous extract from Grifola frondosa, a minor fraction of an aqueous extract from Hericium erinaceus and a minor fraction of an aqueous extract from Cistus creticus.

Accordingly, such suitable compositions may for example contain a major fraction of an aqueous extract from Agaricus blazei Murill mycelium, and a minor fraction of aqueous extract from Grifola frondosa mycelium, a minor fraction of an aqueous extract from Hericium erinaceus stalks and a minor fraction of an aqueous extract from Cistus creticus.

As used herein, a major fraction means more than or equal to 50 vol %. Accordingly, a major fraction can for example be in the range of 50 to 95 vol %, 60 to 80 vol %, 55 to 75 vol % etc. As used herein, a minor fraction means less than 50 vol %. Accordingly, a minor fraction can for example be in the range of 1 to 10 vol %, 5 to 20 vol %, 10 to 40% etc.

Accordingly, such suitable compositions may for example contain 10 to 70 wt % of an aqueous extract from Agaricus blazei Murill mycelium, and 10 to 70 wt % of aqueous extract from Grifola frondosa mycelium, 0 to 40 wt % of an aqueous extract from Hericium erinaceus stalks and 0 to 10 wt % of an aqueous extract from Cistus creticus.

In particular, the present disclosure provides extracts like Andosan wherein 0.5 to 10 wt % of Cistus creticus powder is dissolved. Such compositions may for example comprise 1 to 8 wt % dissolved Cistus creticus powder or 1 to 5 wt % dissolved Cistus creticus powder.

The present disclosure provides compositions comprising
10 to 70 wt % of an aqueous extract from Agaricus blazei Murill mycelium,
10 to 70 wt % of aqueous extract from Grifola frondosa mycelium,
1 to 40 wt % of an aqueous extract from Hericium erinaceus stalks, and
1 to 10 wt % of a dissolved powder from Cistus creticus.

The present disclosure provides compositions comprising
50 to 90 wt % of an aqueous extract from Agaricus blazei Murill mycelium,
20 to 50 wt % of aqueous extract from Grifola frondosa mycelium,
1 to 10 wt % of an aqueous extract from Hericium erinaceus stalks, and
1 to 10 wt % of a dissolved powder from Cistus creticus.

The present disclosure provides compositions comprising
50 to 90 wt % of an aqueous extract from Agaricus blazei Murill mycelium,
20 to 50 wt % of aqueous extract from Grifola frondosa mycelium,
1 to 10 wt % of an aqueous extract from Hericium erinaceus stalks, and
1 to 5 wt % of a dissolved powder from Cistus creticus.

All the compositions herein may be filtered as appropriate for their intended use.

All the compositions herein may comprise propolis extracts, i.e. extracts obtainable from propolis by conventional methods like maceration or Soxhlet extraction. Such methods are described by Bankova et al 2021, Journal of Apicultural Research, 60:5, 734-743.

The compositions mentioned above may contain dry matter in the range of 0.1 to 100 g per 1, 0.5 to 50 g per 1, 1 to 30 g per 1, 2 to 10 g per 1 or 3 to 5 g per 1.

The pH of the compositions in the present disclosure, as conventionally measured by 25° C., may be in the range of 5 to 8, 5.5 to 7.5, 6.5 to 8 or 6 to 7.

As mentioned in U.S. Ser. No. 10/905,7288, in vivo studies have demonstrated an immunological stabilizing and an anti-inflammatory effect of AndoSan™ both in healthy volunteers and in patients with inflammatory bowel disease when given orally. Accordingly, based on the experimental data in the present disclosure, oral administration of the compositions herein may provide an antiviral effect against coronavirus infections. However, as coronaviruses are known to infect cells in the respiratory tract, intranasal or pulmonary administration may also be effective.

Intranasal administration offers many advantages over common routes such as the oral route, as it is non-invasive and easily accessible for the administration of drugs. Further, intranasally administered compositions may be rapidly absorbed and exhibit a fast onset of action due to the rich vasculature in the submucosa. Furthermore, the avoidance of the metabolic first pass effect can lead to a higher bioavailability compared to oral administration. Intranasal administration may be achieved by suitable spraying device.

Pulmonary administration can be achieved by metered dose inhalers, nebulizers or other suitable devices like spraying devices.

Several metered dose inhalers are known. A metered dose inhaler is usually designed for administration and delivery of a specific dose of a pharmaceutical formulation to the lungs which upon operation by the patient, provides a short burst of aerosolized formulation, which is inhaled by the patient.

A nebulizer is a device used to administer a liquid formulation in the form of a mist which is continuously inhaled by tidal breathing. Typical administration time is 20 minutes of continuous and steady breathing through a mask. When administering a pharmaceutical composition using a nebulizer, the patients may inhale the aerosols formed by the device by tidal breathing.

It is well known that pulmonary penetration is predominantly depending on particle size and inhalation flow rate. Efficient pulmonary penetration often requires particle size in the range 1 to 5 μm. Larger particles tend to impact the oropharynx and large airways, but particles less than 1 μm tend to being exhaled. The compositions herein may be administered by inhalation of monodisperse droplets formed by aerosolization, and said droplets may for example have a mass median aerodynamic diameter of 3 to 7 μm using a suitable metered dose inhaler providing a flow of 5 to 50 L/min.

Alternatively, the compositions herein may be sprayed onto surfaces in the oral cavity. Such local administration may have several advantages, and it may be considered to be less invasive than oral administration for systemic exposure. Sprays for oral administration may comprise larger droplets compared to sprays intended for pulmonary administration. Suitable median droplet size for sprays intended for surfaces in the oral cavity may be in the range of 100 to 3000 μm, 200 to 2000 μm 300 to 1500 μm, 400 to 1200 μm. Devices for providing such sprays are well known. One such example is the Mycoshield® spray provided by Host Defense® Mushrooms™. Compositions intended for spraying onto surfaces in the oral cavity may comprise optional ingredients like sweeteners or desired flavor. They may also contain pH-adjusting agents and/or other excipients. The compositions disclosed herein may thus be suitable for local administration to surfaces in the oral cavity. Such local administration may be either medical use or non-medical use. For example, even in cases where no therapeutic effect nor prophylactic effect would be demonstrated for a composition disclosed herein when sprayed on surfaces in the oral cavity, it could still be used for other non-medical purposes like well-being, nutrition, placebo etc.

The compositions herein may be pharmaceutically acceptable aqueous solutions, capsules comprising them or lyophilized powders obtained from the pharmaceutically acceptable aqueous solutions.

“Pharmaceutically acceptable composition”, as used herein, means any composition suitable and intended for in vivo use, for example administration to a patient or a subject in need thereof. As used herein, the terms “patient” and “subject” are interchangeable and refer to any human or animal individual who is receiving a composition as described herein. Such animals may include pets, farm animals and other livestock.

We thus provide a pharmaceutically acceptable composition comprising
a. an extract from Agaricus blazei
b. an extract from Grifola frondosa, and
c. an extract from Hericium erinaceus
for use in treatment of coronavirus infections, e.g. therapeutic or prophylactic treatment of SARS, MERS or COVID-19.

As a separate general aspect, we also provide a pharmaceutically acceptable composition comprising an aqueous extract from Agaricus blazei for use in treatment of coronavirus infections.

As a separate general aspect, we also provide a pharmaceutically acceptable composition comprising an aqueous extract from Grifola frondosa for use in treatment of coronavirus infections.

As a separate general aspect, we also provide a pharmaceutically acceptable composition comprising an aqueous extract from Hericium erinaceus for use in treatment of coronavirus infections.

As a separate general aspect, we also provide a pharmaceutically acceptable composition comprising an aqueous extract from Agaricus blazei for use in treatment of SARS-CoV-2 infections.

As a separate general aspect, we also provide a pharmaceutically acceptable composition comprising an aqueous extract from Grifola frondosa for use in treatment of SARS-CoV-2 infections.

As a separate general aspect, we also provide a pharmaceutically acceptable composition comprising an aqueous extract from Hericium erinaceus for use in treatment of SARS-CoV-2 infections.

However, the compositions herein may also be food supplements, nutraceuticals, functional food in the form of aqueous solutions, capsules comprising them or lyophilized powders obtained from the aqueous solutions. Such non-drug compositions, even if not directly indicated for treatment of coronavirus infections, may reduce the risk of getting a coronavirus infection. This effect may partly arise from prevention of the interaction between the Spike S1 protein and ACE2, but it may also partly arise from other systemic or local immune-system modulating effects. These effects may in turn reduce the coronaviruses ability to infect and/or replicate in the host. Accordingly, consuming the compositions herein, especially through long-term daily consumption, may improve the immune system readiness with respect to coronavirus challenges, such as the SARS-CoV-2 pandemic.

In the examples 1 and 2 below, a Composition A was tested. Composition A was a solution comprising an aqueous extract from Agaricus subrufescens mycelium, and a minor fraction of aqueous extract from Grifola frondosa mycelium, and a minor fraction of an aqueous extract from Hericium erinaceus stalks. It also contained a propolis extract as a conservative. The dry matter content was ca 4 g per 1, and the pH was around 7.

### Example 1

The effect of Composition A on the infection of cells by SARS-CoV-2 pseudotyped virus.

HEK-Blue™ hACE2 cells that express high levels of the human (h)ACE2 receptor at their cell surface were obtained from InvivoGen. Cells were cultured in Dulbecco's modified Eagle's medium (DMEM) supplemented with 10% (v/v) foetal bovine serum, at 37° C. in a humidified, CO2-controlled (5%) incubator.

SARS-CoV-2-pseudotyped virus stocks were produced in 293T cells by co-transfection of pNL4-3. Luc.R-E-together with pLV-SARS2-S-d19 plasmid that encodes the Spike (S) gene (codon-optimized) from the original SARS-CoV-2 Wuhan-Hu-, using the calcium phosphate transfection system. Supernatants, containing virus stocks, were harvested 48 h post-transfection and were centrifuged 5 min at 500 g to remove cell debris, and stored at −80° C. until use.

It was first established that SARS-CoV-2 infected HEK-cells expressing ACE2 (HEK-Blue™ hACE2 cells). Other HEK-cells were far less infected. The infection was largely neutralized with a specific anti-COVID-19 monoclonal antibody.

The HEK-Blue™ hACE2 cells were then incubated with Composition A before the addition of the virus stocks. We found Composition A inhibited the SARS-CoV-2 pseudotyped virus from infecting HEK-Blue-ACE2.

HEK-Blue™ hACE2 cells (10⁵/ml) were plated on a 24-well plate and incubated with three non-cytotoxic concentrations of the test substances for 30 min. Immediately after this step the cells were inoculated with virus stocks for 1 h, then washed twice with PBS and incubated for additional 23 h. Finally, the cells were washed twice in PBS and lysed in 25 mM Tris-phosphate pH 7.8, 8 mM MgCl₂, 1 mM DTT, 1% Triton X-100, and 7% glycerol during 15 μm at RT. Then, the lysates were spun down, and the supernatant used to measure luciferase activity using an Autolumat LB 9510 (Berthold, Bad Wildbad, Germany) following the instructions of the luciferase assay kit (Promega). The results are represented as the % of activation (considering the infected and untreated cells 100% activation) or RLU. Upon integration into host chromosomes, this recombinant virus expresses the firefly luciferase gene and consequently luciferase activity in infected cells correlates with the rate of viral replication. Thus, high luciferase activity levels were detected 24 h after cellular infection with the SARS-CoV-2 pseudotyped clone. As a positive control of the infective process a neutralizing anti-SARS-CoV-2 RBD mAb (Clone B38 from Invivogen) was used.

### Example 2

The effect of Composition A on the binding of SARS-CoV-2 Spike S1 protein to ACE2 ex vitro.

We tested the effects of Composition A in two different screenings assays analyzing the effects of test items on the interaction between ACE2 and the Spike S1 protein. The ACE2:SARS-CoV-2 Spike S1 Inhibitor Screening Assay Kit is designed for screening and profiling inhibitors of this interaction.

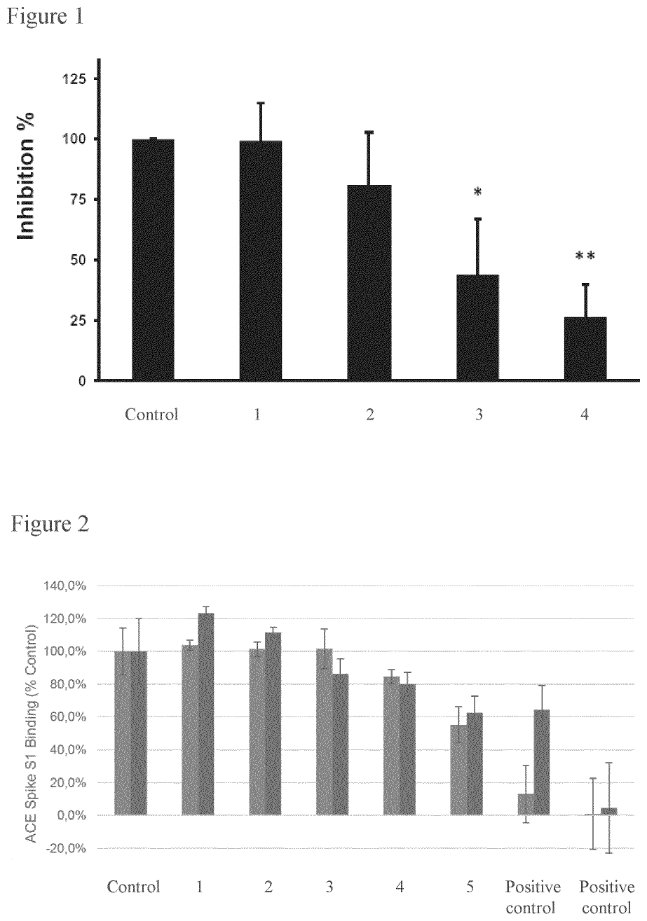

As visualized in FIG. 2 and the table below, Composition A shows inhibitory effects in duplicate assays measuring the interaction between ACE2 and Spike S1 protein.

| | 0.01 vol % | 0.1 vol % | 1 vol % | 5 vol % | 10 vol % | | |
|---|---|---|---|---|---|---|---|
| | Composition | Composition | Composition | Composition | Composition | 10 nM | 100 nM |
| Control | A | A | A | A | A | antibody | antibody |
| 100.0% | 103.9% | 101.3% | 101.5% | 84.6% | 55.3% | 13.1% | 1.0% |
| 100.0% | 123.3% | 111.5% | 86.3% | 80.1% | 62.6% | 64.3% | 4.5% |

### Example 3

A spraying device comprising a suitable nozzle and a container can be loaded with 50 ml of an aqueous sterile solution with neutral pH comprising aqueous extracts from Agaricus blazei, Grifola frondosa, Hericium erinaceus and Cistus creticus. The spraying device can provide a spray comprising a major fraction of droplets with a particle size in the range of 1 to 5 μm. Such spray will be suitable to reach pulmonary surfaces if inhaled.

### Example 4

A spraying device comprising a suitable nozzle and a container can be loaded with 100 ml of an aqueous sterile solution with pH 6 comprising aqueous extracts from Agaricus blazei, Grifola frondosa, Hericium erinaceus and Cistus creticus. The spraying device can provide a spray comprising a major fraction of droplets with a particle size in the range of 100 to 1000 μm. Such spray will be suitable for administration to the oral cavity.

### Example 5

The following extracts were provided: 1) Andosan (liquid; Lot: 032021C), 2) Extr. Cisti e herb. Aquos. Sicc Ecce20 (FinzelbergLot: 22000598). The Cistus creticus powder was dissolved in DMSO and the stock was 100 mg/ml. The final amount of DMSO in the cell cultures did not affect the cellular assays or the NA activity. The test samples contained AndoSan™ in combination (2:1 w/w ratio) with the Cistus creticus extract.

### NA-XTD™ Neuraminidase Assay

The NA-XTD™ neuraminidase assay is a highly sensitive method to detect neuraminidase enzyme activity using NA-XTD™ chemiluminescent substrate. This chemiluminescent assay provides a highly sensitive and rapid convenient assay for screening assays aimed at the identification and development of new neuraminidase inhibitors (Thermo Fisher, catalog number 4457535). To perform the assay, the test samples were incubated during 20 min at 37° C. with 0,312×10⁶ U/mL Neuraminidase from Influenza A Virus (R&D, reference: 4858-NM-005) and then incubated for 30 min with NA-Star substrate. After that time NA-XTD™ accelerator was added and plates were placed in a microplate reader (Tristar LB-941, Berthold Tech). Light signal intensity is measured using a 2 sec/well read. A background signal will be subtracted from each data point. All experiments were repeated twice in triplicate, and results are expressed as the means±sd. In order to validate the technique, the neuraminidase inhibitors Zanamivir 1.28 nM and the extract solvent (DMSO) were included as controls in all the assays.

### Cell Cultures

HEK-Blue™ hACE2 cells which express high levels of the human (h)ACE2 receptor at their cell surface were obtained from InvivoGen. ACE2 (angiotensin I-converting enzyme-2) is a type I membrane protein that is established as the host receptor for the Spike (S) protein of severe acute respiratory syndrome coronaviruses, SARS-CoV, and SARS-CoV-2. Cells were cultured in Dulbecco's modified Eagle's medium (DMEM) supplemented with 10% (v/v) foetal bovine serum, at 37° C. in a humidified, CO₂-controlled (5%) incubator.

Production of VSV-pseudotyped recombinant lentiviruses SARS-Cov-2-pseudotyped virus stocks were produced in 293T cells by co-transfection of pNL4-3. Luc.R-E-together with pLV-SARS2-S-d19 plasmid that encodes the Spike (S) gene (codon optimized) from the original SARS-CoV-2 Wuhan Hu-, using the calcium phosphate transfection system. Supernatants, containing virus stocks are harvested 48 h post-transfection, centrifuged 5 min at 500 g to remove cell debris and stored at −80° C. until use.

### SARS-CoV-2-Pseudotyped Infection Assay

HEK-Blue™ hACE2 cells (10⁵/ml) were plated on a 24-well plate and incubated with different non-cytotoxic concentrations of the test substances for 30 or 60 min. After this time the cells will be inoculated with 200 μL virus stocks and 24 h later cells were washed twice in PBS and lysed in 25 mM Tris-phosphate pH 7.8, 8 mM MgCl₂, 1 mM DTT, 1% Triton X-100, and 7% glycerol during 15 μm at RT. Then, the lysates were spun down, and the supernatant used to measure luciferase activity using an Autolumat LB 9510 (Berthold, Bad Wildbad, Germany) following the instructions of the luciferase assay kit (Promega). The results are represented as the % of activation (considering the infected and untreated cells 100% activation) or RLU. Upon integration into host chromosomes, this recombinant virus expresses the firefly luciferase gene and consequently luciferase activity in infected cells correlates with the rate of viral replication. Thus, high luciferase activity levels were detected 24 h after cellular infection with the SARS-CoV-2 pseudotyped clone. As a positive control a neutralizing anti-ARS-CoV-2 RBD mAb (Clone B38 from Invivogen) was used.

### Cytotoxicity Assay

To select non-cytotoxic concentrations for studies on SARS-CoV-2 infection HEK-Blue™ hACE2 cells (10³/well) were incubated in 96-well plates and treated with increasing concentrations of the test samples and toxicity was evaluated by the confluence software of the Incucyte apparatus (Sartorius).

1. A method of treating a coronavirus infection in a subject in need thereof, comprising administering a pharmaceutically acceptable composition comprising
a) an extract from Agaricus blazei,
b) an extract from Grifola frondosa, and/or
c) an extract from Hericium erinaceus.

2. The method according to claim 1, wherein the composition comprises
a) an extract from Agaricus blazei,
b) an extract from Grifola frondosa, and
c) an extract from Hericium erinaceus.

3-14. (canceled)

15. A composition comprising
a. an extract from Agaricus blazei,
b. an extract from Grifola frondosa,
c. an extract from Hericium erinaceus, and
d. an extract from Cistus creticus.

16. The composition according to claim 15, wherein the composition comprises an extract obtained by aqueous extraction of a mixture comprising 50 to 90 wt % mycelium from Agaricus blazei.

17. The composition according to claim 15, wherein the composition comprises an extract obtained by aqueous extraction of a mixture comprising 10 to 50 wt % mycelium from Grifola frondosa.

18. The composition according to claim 15, wherein the composition comprises an extract obtained by aqueous extraction from Hericium erinaceus stalks.

19. The composition according to claim 15, wherein the extracts are obtained by aqueous extraction of mycelium from Agaricus blazei, mycelium from Grifola frondosa and stalks from Hericium erinaceus.

20. The composition according to claim 15, wherein the pH is the range of 5 to 8.

21. The composition according to claim 15, wherein the composition is aqueous.

22. The composition according to claim 15, wherein the composition is sterile.

23. The composition according to claim 15, wherein the composition is suitable for intranasal administration, pulmonary administration or for administration to the oral cavity.

24. The composition according to claim 15, wherein the composition is classified as novel food, food supplement, nutraceutical or functional food.

25. A device for oral, intranasal or pulmonary administration comprising the composition according to claim 15.

26. The device according to claim 25, comprising a nozzle configured to provide a spray of liquid particles able to reach pulmonary surfaces.

27. The device according to claim 25, comprising a nozzle configured to provide a spray of liquid particles able to reach intranasal surfaces.

28. The device according to claim 25, comprising a nozzle configured to provide a spray of liquid particles able to reach surfaces in the oral cavity.

29. (canceled)
